(19) United States
(12) Patent Application Publication
(10) Pub. No.: US 2015/0366847 A1
(43) Pub. Date: Dec. 24, 2015

(54) COMBINATION OF GERANYLGERANYLACETONE AND IBUDILAST AND METHODS OF USING SAME

(71) Applicant: MediciNova, Inc., La Jolla, CA (US)

(72) Inventors: Kazuko MATSUDA, Beverly Hills, CA (US); Yuichi IWAKI, La Jolla, CA (US)

(73) Assignee: MediciNova, Inc., La Jolla, CA (US)

(21) Appl. No.: 14/742,274

(22) Filed: Jun. 17, 2015

Related U.S. Application Data
(60) Provisional application No. 62/015,257, filed on Jun. 20, 2014.
(60) Provisional application No. 62/084,397, filed on Nov. 25, 2014.

(51) Int. Cl. A61K 31/437; A61K 31/121

(52) U.S. Cl. CPC A61K 31/437; A61K 31/121

(57) ABSTRACT

The present invention relates generally to methods for treating progressive neurodegenerative diseases, including their progressive forms. In particular, the present invention pertains to methods of treating or preventing progressive neurodegenerative diseases and its associated symptoms by administration of a combination of geranylgeranylacetone (teprenone) and ibudilast (3-isobutyryl-2-isopropylpyrazolo[1,5-a]pyridine), or pharmaceutically acceptable salts thereof.

### CROSS REFERENCE TO RELATED APPLICATIONS

This application the benefit, under 35 U.S.C. §119(e), of U.S. Provisional Application Ser. No. 62/015,257, filed Jun. 20, 2014, and U.S. Provisional Application Ser. No. 62/084,397, filed Nov. 25, 2014, the entire contents of which are incorporated by reference herein.

### FIELD OF THE INVENTION

The present invention relates generally to methods for treating neurodegenerative diseases, including those which are of the progressive variety. For example, the present invention pertains to methods of treating or preventing progressive neurodegenerative diseases and its associated symptoms by administration of a combination of geranylgeranylacetone (teprenone) and ibudilast (3-isobutyryl-2-isopropylpyrazolo[1,5-a]pyridine).

### BACKGROUND OF THE INVENTION

Ibudilast is a small molecule drug (molecular weight of 230.3) having the structure shown below.

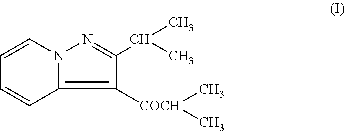

Ibudilast is also found under ChemBank ID 3227, CAS #50847-11-5, and Beilstein Handbook Reference No. 5-24-03-00396. Its molecular formula corresponds to C₁₄H₁₈N₂O. Ibudilast is also known by various chemical names including 2-methyl-1-(2-(1-methylethyl)pyrazolo(1,5-a)pyridin-3-yl)1-propanone; 3-isobutyryl-2-isopropylpyrazolo (1,5-a)pyridine]; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and MN-166. Its brand name is Ketas®. Ibudilast, as referred to herein, is meant to include any and all pharmaceutically acceptable salt forms thereof, prodrug forms (e.g., the corresponding ketal), solvates, and the like, as appropriate for use in its intended formulation for administration.

Ibudilast has been widely used in Japan for relieving symptoms associated with ischemic stroke or bronchial asthma. Marketed indications for ibudilast in Japan include its use as a vasodilator, for treating allergy, eye tissue regeneration, ocular disease, and treatment of allergic ophthalmic disease (Thompson Current Drug Reports). US Patent Application Publication No. 20060160843 discloses ibudilast for the treatment of intermittent and short term pain, however this is not pain related to a progressive form of a neurodegenerative disease. US Patent Application Publication No. 2009/0062330 discloses the treatment of progressive neurodegenerative diseases by the administration of ibudilast. This publication generally discloses that ibudilast may be administered in a combination treatment along with an additional agent effective for treating progressive neurodegenerative diseases, but does not provide any guidance on the choice of the additional agent.

Geranylgeranyl acetone (GGA) has the formula:

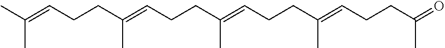 [SMILES: CC(=O)CC/C=C(\C)CC/C=C(\C)CC/C=C(\C)CCC=C(C)C]

and is marketed as an antiulcer agent in Japan. GGA, alternatively known as teprenone, is also reported to have neuroprotective and related effects. See, for example, PCT Pat. App. Pub. Nos. WO 2012/031028, WO 2013/052148, and WO 2013/130654, each of which is incorporated herein by reference in its entirety. It has also been reported that GGA is an inducer of heat shock protein 70 (HSP70) in mice subjected to an occlusion of the middle cerebral artery.

### SUMMARY OF THE INVENTION

The present invention relates to a novel approach to treating a neurodegenerative disease or disorder, including progressive forms, and is based on the administration of a combination of ibudilast and geranylgeranylacetone.

Accordingly, in one embodiment, the present invention is a method of alleviating negative effects of a neurodegenerative disease or disorder in a human patient suffering therefrom, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In one embodiment, the invention is a method of slowing progression of disease in a patient diagnosed with a chronic neurodegenerative disease, comprising administering to the patient:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In another embodiment, the invention is a method of treating a patient diagnosed with a neurodegenerative disease or disorder, comprising administering to the patient:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In one embodiment, the neurodegenerative disease or disorder is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, primary lateral sclerosis, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, but not relapsing, remitting multiple sclerosis, frontotemporal dementia, Wilson's disease, progressive neuropathic pain, ischemia caused by stroke, traumatic brain injury, or spinal cord injury.

In one embodiment, the neurodegenerative disease is of the progressive type.

In another embodiment, the neurodegenerative disease or disorder is Alzheimer's disease, Parkinson's disease, amyotrophic lateral sclerosis (ALS), or multiple sclerosis.

In one embodiment, the invention is a method of reducing a volume of an infarct (an area of necrosis in a tissue or organ resulting from obstruction of the local circulation by a thrombus or embolus) in a patient suffering from an ischemia, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof, in which a volume of an infarct in the treated patient is reduced compared to a volume of an infarct in a control patient.

In some embodiments, the ibudilast and GGA, or pharmaceutically acceptable salts thereof, are administered orally.

In one embodiment, ibudilast and GGA, or pharmaceutically acceptable salts thereof, are administered in separate dosage forms. In another embodiment, the ibudilast or pharmaceutically acceptable salt thereof, the GGA or a pharmaceutically acceptable salt thereof, are administered in the same dosage form.

In one embodiment, the ibudilast or a pharmaceutically acceptable salt thereof is administered in an amount from about 100 to about 4,000 mg/day, divided into one, two, or three portions. In another embodiment, the GGA or pharmaceutically acceptable salt thereof is administered in an amount from about 1 mg/kg to about 1000 mg/kg of the patient.

In one embodiment, the invention is a composition for oral administration, comprising:
(a) ibudilast or a pharmaceutically acceptable salt thereof,
(b) GGA, and
(c) optionally, a pharmaceutically acceptable excipient or carrier.

In one embodiment, the composition comprises from about 100 to about 4,000 mg of ibudilast or a pharmaceutically acceptable salt thereof and from about 50 mg to about 4,000 mg of GGA or a pharmaceutically acceptable salt thereof.

In one embodiment, the composition is an oral tablet or capsule. In another embodiment, the composition is an oral liquid dosage form.

### BRIEF DESCRIPTION OF THE DRAWINGS

FIG. 1 graphically illustrates comparative life span in Sod1 mutants (Sod1⁻) and wt (Sod1+). Maximum life span of mutants is 25-30 days compared to 70-80 days for controls; recovering on life span curve can indicate positive compound activity.

FIG. 2 graphically illustrates high (% viability) sensitivity of SOD1-null adults compared to a wild stock after exposition of adult flies to 2 mmol of paraquat. Resistance to paraquat treatment can indicate positive activity of the compound tested.

### DETAILED DESCRIPTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20th Ed.; FDA's Orange Book, Goodman & Gilman The Pharmacological Basis of Therapeutics, J. Griffith Hardman, L. L. Limbird, A. Gilman, 11th Ed., 2005, The Merck Manual, 18th edition, 2007, and The Merck Manual of Medical Information 2003.

All publications cited herein, including internet articles, the FDA Orange Book (available on the FDA's website), books, handbooks, journal articles, patents and patent applications, whether supra or infra, are hereby incorporated by reference in their entirety.

### DEFINITIONS

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

“Administering” or “administration of” a drug to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“Comprising” shall mean that the methods and compositions include the recited elements, but not exclude others. “Consisting essentially of” when used to define methods and compositions, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

“Effective amount” of a compound utilized herein is an amount that, when administered to a patient treated as herein, will have the intended therapeutic effect, e.g., alleviation, amelioration, palliation or elimination of one or more manifestations of the medical condition in the patient. The full therapeutic effect does not necessarily occur by administration of one dose (or dosage), and may occur only after administration of a series of doses. Thus, an effective amount may be administered in one or more administrations.

“Pharmaceutically acceptable” refers to non-toxic and suitable for administration to a patient, including a human patient.

“Pharmaceutically acceptable salts” refer to salts that are non-toxic and are suitable for administration to patients. “Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Treating” a medical condition or a patient refers to taking steps to obtain beneficial or desired results, including clinical results. For purposes of the various aspects and embodiments of the present invention, beneficial or desired clinical results include, but are not limited to, reduction, alleviation, or amelioration of one or more manifestations of or negative effects of ALS, PLS or familial ALS, improvement in one or more clinical outcomes, diminishment of extent of sclerosis, delay or slowing of sclerosis progression, amelioration, palliation, or stabilization of the scleroses state, and other beneficial results described herein.

By “neurodegenerative disease” means any neurodegenerative disease that causes the loss of structure or function of neurons in the nervous system of an individual, including death of neurons. Neurodegenerative disease includes progressive neurodegenerative diseases, in which the symptoms worsen over time. In certain instances, the symptoms worsen at a gradual rate. Examples of progressive neurodegenerative diseases include Alzheimer's Disease, Parkinsonism and amyotrophic lateral sclerosis. An example of a neurodegenerative disease is a relapsing and remitting form of multiple sclerosis (MS). MS also exhibits progressive forms.

The term “central nervous system” or “CNS” includes all cells and tissue of the brain and spinal cord of a vertebrate. Thus, the term includes, but is not limited to, neuronal cells, glial cells, astrocytes, cerebrospinal fluid (CSF), interstitial spaces and the like.

The terms “subject,” “individual,” or “patient” are used interchangeably herein and refer to a vertebrate, preferably a mammal. Mammals include, but are not limited to, murines, rodents, simians, humans, farm animals, sport animals and pets.

The terms “pharmacologically effective amount” or “therapeutically effective amount” of a composition or agent, as provided herein, refer to a nontoxic but sufficient amount of the composition or agent to provide the desired response, such as a reduction, alleviation, or reversal of the symptoms of neurodegenerative diseases. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “about,” particularly in reference to a given quantity, is meant to encompass deviations of plus or minus five percent.

As stated previously, a reference to any one or more of the herein-described drugs, in particular ibudilast, is meant to encompass, where applicable, any and all enantiomers, mixtures of enantiomers including racemic mixtures, prodrugs, pharmaceutically acceptable salt forms, hydrates (e.g., monohydrates, dihydrates, etc.), solvates, different physical forms (e.g., crystalline solids, amorphous solids), metabolites, and the like.

In some embodiments, the GGA used in a composition of the present invention is a synthetic 5-trans isomer compound of formula III:

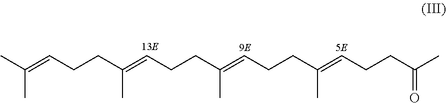 [SMILES: CC(=O)CC/C=C(\C)CC/C=C(\C)CC/C=C(\C)CCC=C(C)C]

wherein III is at least 80% in the 5E, 9E, 13E configuration. In some embodiments, the invention utilizes a compound of formula III wherein at least 85%, or at least 90%, or at least 95%, or at least 96%, or at least 97%, or at least 98%, or at least 99%, or at least 99.5%, or at least 99.9% is in the 5E, 9E, 13E configuration. In some embodiments the invention for the compound of formula III does not contain any of the cis-isomer of GGA.

In other embodiments, the GGA used in a composition of the present invention is a synthetic 5-cis isomer compound of formula IV:

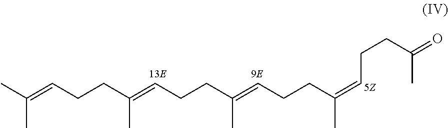 [SMILES: CC(=O)CC/C=C(/C)CC/C=C(\C)CC/C=C(\C)CCC=C(C)C]

wherein IV is at least 75% in the 5Z, 9E, 13E configuration. In certain embodiments, the invention utilizes a compound of formula IV wherein IV is at least 80% in the 5E, 9E, 13E configuration, or alternatively, at least 85%, or at least 90%, or at least 95%, or at least 96%, or at least 97%, or at least 98%, or at least 99%, or at least 99.5%, or at least 99.9% in the 5E, 9E, 13E configuration. In some embodiments of the invention, the compound of formula VII does not contain any of the trans-isomer of GGA.

The configuration of compounds can be determined by methods known to those skilled in the art such as chiroptical spectroscopy and nuclear magnetic resonance spectroscopy.

### Methods of Treatment

In one embodiment, the invention is a method of alleviating negative effects of a neurodegenerative disease or disorder in a human patient suffering therefrom, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In one embodiment, the invention is a method of slowing progression of disease in a patient diagnosed with a chronic neurodegenerative disease, comprising administering to the patient:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In another embodiment, the invention is a method of treating a patient diagnosed with a neurodegenerative disease or disorder, comprising administering to the patient a composition comprising:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

In one embodiment, the neurodegenerative disease or disorder compromises the nervous system. In any case, the ibudilast and GGA may be administered together or separately. The ibudilast and GGA may be administered at the same time or at different times.

In another embodiment, the neurodegenerative disease or disorder is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, primary lateral sclerosis, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, but not relapsing, remitting multiple sclerosis, frontotemporal dementia, Wilson's disease, progressive neuropathic pain, ischemia caused by stroke, traumatic brain injury, or spinal cord injury.

In one embodiment, the neurodegenerative disease is of the progressive type.

In a particular embodiment, the neurodegenerative disease or disorder is Alzheimer's disease, Parkinson's disease, amyotrophic lateral sclerosis (ALS), or multiple sclerosis.

In one embodiment, the invention is a method of reducing a volume of an infarct (an area of necrosis in a tissue or organ resulting from obstruction of the local circulation by a thrombus or embolus) in a patient suffering from an ischemia, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof,
   in which a volume of an infarct in the treated patient is reduced compared to a volume of an infarct in a control patient.

### Methods of Administration

In some embodiments, the present invention comprises administering ibudilast and GGA, or pharmaceutically acceptable salts thereof, either systemically or centrally (e.g., by intrathecal administration, i.e., into the cerebrospinal fluid surrounding the spinal cord). In a other embodiments, the present invention comprises administering ibudilast and GGA, or pharmaceutically acceptable salts thereof, systemically, e.g., via parenteral, enteral, oral, intravenous, intranasal, sublingual or other systemic routes, to a human, subject for the treatment of progressive neurodegenerative diseases.

In a particular embodiment, the present invention comprises administering ibudilast and GGA, or pharmaceutically acceptable salts thereof, orally.

In one embodiment, the invention comprises administering ibudilast and GGA, or pharmaceutically acceptable salts thereof, as a single combination composition. In terms of patient compliance and ease of administration, such an approach is preferred, since patients are often adverse to taking multiple pills or dosage forms, often multiple times daily, over the duration of treatment. Alternatively, in another embodiment, the invention comprises administering ibudilast and GGA, or pharmaceutically acceptable salts thereof, as separate dosage forms. In embodiments in which ibudilast and GGA are administered as separate dosage forms and co-administration is required, ibudilast and GGA may be administered simultaneously, sequentially in any order, or separately.

### Doses and Dosage Forms

In one embodiment, the invention is a composition comprising:
(a) ibudilast or a pharmaceutically acceptable salt thereof,
(b) GGA or a pharmaceutically acceptable salt thereof, and
(c) optionally, a pharmaceutically acceptable excipient or carrier.

In one embodiment, the invention is a composition, comprising:
(a) from about 100 to about 4,000 mg/day of ibudilast or a pharmaceutically acceptable salt thereof,
(b) from about 50 mg to about 4,000 mg of GGA or a pharmaceutically acceptable salt thereof, and
(c) optionally, a pharmaceutically acceptable excipient or carrier.

In other embodiments, the invention is a composition, comprising:
(a) a therapeutically effective amount of ibudilast or a pharmaceutically acceptable salt thereof,
(b) a therapeutically effective amount of GGA, and
(c) optionally, a pharmaceutically acceptable excipient or carrier,
   wherein the composition is effective to alleviate the negative effects of a neurodegenerative disease or disorder in a human patient suffering therefrom.

Therapeutically effective amounts can be determined by those skilled in the art, and will be adjusted to the requirements of each particular case. Effective dosage levels of ibudilast can vary from about 100 to about 4000 mg per day. In one embodiment, the daily dosage range is 250 to 2,000 mg, given in one, two, or three portions. In one embodiment, the daily dosage range of ibudilast is 100 to 500 mg, such as 100, 200, 300, 400, or 500 mg given in one, two, or three portions. In one embodiment, the daily dosage range of ibudilast is about 250 to about 2,000 mg, such as 250, 500, 750, 1,000, 1,250, 1,500, 1,750, or 2,000 mg given in one, two, or three portions. In one embodiment, the daily dosage range of ibudilast is from about 1000 to about 4,000 mg, such as about 1,000, about 2,000, about 3,000, or about 4,000 mg, given in one, two, or three portions. In another embodiment, the dosage is about 1000 mg twice a day. In other embodiments, suitable dosages of ibudilast include about 1000 mg four times a day, about 1000 mg twice a day, and about 750 mg three times a day.

An effective dose of GGA in a composition of the present invention is from about 0.1 mg/kg/day to about 4,000 mg/kg/day, or from about 1 mg/kg/day to about 50 mg/kg/day, or from about 1 mg/kg/day to about 25 mg per kg/day. In some other embodiments, the effective amount of GGA is from about 10 mg/kg/day to about 100 mg/kg/day, about 20 mg/kg/day to about 90 mg/kg/day, about 30 mg/kg/day to about 80 mg/kg/day, about 40 mg/kg/day to about 70 mg/kg/day, or about 50 mg/kg/day to about 60 mg/kg/day. In still some other embodiments, the dose of GGA is from about 100 mg/kg/day to about 1000 mg/kg/day.

Actual amounts will depend on the circumstances of the patient being treated. As those skilled in the art recognize, many factors that modify the action of the active substance will be taken into account by the treating physician such as the age, body weight, sex, diet and condition of the patient, the time of administration, the rate and route of administration. Optimal dosages for a given set of conditions can be ascertained by those skilled in the art using conventional dosage determination tests.

The compounds utilized herein can be formulated in any pharmaceutically acceptable form, including liquids, powders, creams, emulsions, pills, troches, suppositories, suspensions, solutions, and the like. Compositions according to the present invention will ordinarily be formulated with one or more pharmaceutically acceptable ingredients in accordance with known and established practice. In general, tablets are formed utilizing a carrier such as modified starch, alone or in combination with carboxymethyl cellulose (Avicel), for example at about 10% by weight. The formulations are compressed at from 1,000 to 3,000 pounds pressure in the tablet forming process. The tablets preferably exhibit an average hardness of about 1.5 to 8.0 kp/cm², preferably 5.0 to 7.5 kp/cm². Disintegration time varies from about 30 seconds to about 15 or 20 minutes.

Compositions for oral use can be provided as hard gelatin capsules wherein the therapeutically active compounds utilized herein are mixed with an inert solid diluent such as calcium carbonate, calcium phosphate or kaolin, or as soft gelatin capsules in which the compounds are mixed with an oleaginous medium, e.g., liquid paraffin or olive oil. Suitable carriers include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like.

The compositions of the present invention can be formulated as aqueous suspensions in admixture with pharmaceutically acceptable excipients such as suspending agents including, but not limited to, sodium carboxymethyl cellulose, methylcellulose, hydroxypropylmethyl cellulose, sodium alginate, polyvinylpyrrolidone, gum tragacanth and gum acacia; dispersing or wetting agents such as naturally occurring phosphatide, e.g., lecithin, or condensation products of an alkaline oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Such aqueous suspensions can also contain one or more preservatives, e.g., ethyl- or n-propyl-p-hydroxy benzoate, one or more coloring agents, one or more flavoring agents and one or more sweetening agents, such as glycerol, sorbitol, sucrose, saccharin or sodium or calcium cyclamate.

Suitable compositions of the present invention also include sustained release dosage forms, such as those described in U.S. Pat. Nos. 4,788,055; 4,816,264; 4,828,836; 4,834,965; 4,834,985; 4,996,047; 5,071,646; and, 5,133,974, the contents of which are incorporated herein in their entirety by reference.

Other compositions of the present invention suitable for oral administration include liquid form preparations including emulsions, syrups, elixirs, aqueous solutions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizing, and thickening agents. Solid form preparations may contain, in addition to the active component, colorants, flavors, stabilizers, buffers, artificial and natural sweeteners, dispersants, thickeners, solubilizing agents, and the like.

The compositions of the present invention may be formulated for parenteral administration (e.g., by injection, for example bolus injection or continuous infusion) and may be presented in unit dose form in ampoules, pre-filled syringes, small volume infusion or in multidose containers with an added preservative. The compositions may take such forms as suspensions, solutions, or emulsions in oily or aqueous vehicles, for example as solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compositions of the present invention can be formulated for nasal administration. The solutions or suspensions are applied directly to the nasal cavity by conventional means, for example, with a dropper, pipette or spray. The formulations may be provided in a single or multidose form. The patient can administer an appropriate, predetermined volume of the solution or suspension via a dropper or pipette. A spray may be administered for example by means of a metering atomizing spray pump.

The compositions of the present invention can be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine. The powder carrier will form a gel in the nasal cavity. The powder composition may be presented in unit dose form for example in capsules or cartridges of, for example gelatin or blister packs from which the powder may be administered by means of an inhaler.

The compositions of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges including active agents in a flavored base, usually sucrose and acacia or tragacanth; pastilles including the active ingredient in an inert base such as gelatin and glycerin or sucrose and acacia; and mouthwashes including the active ingredient in a suitable liquid carrier.

The compositions of the present invention may be formulated for administration as suppositories. In such a formulation, a low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compositions of the present invention may be formulated for vaginal administration. Pessaries, tampons, creams, gels, pastes, foams or sprays containing in addition to the active ingredient such carriers as are known in the art to be appropriate.

### Oral Compositions

Oral compositions of the present invention may further comprise one or more pharmaceutically acceptable excipients or carriers. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

A composition of the invention may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in an ibudilast composition of the invention is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

### Modified Release Compositions

When desired, compositions of the present invention can be prepared with enteric coatings adapted for sustained or controlled release administration of the active ingredient. A common type of controlled release formulation that may be used for the purposes of the present invention comprises an inert core, such as a sugar sphere, a first layer, coated with an inner drug-containing second layer, and an outer membrane or third layer controlling drug release from the inner layer.

The cores are preferably of a water-soluble or swellable material, and may be any such material that is conventionally used as cores or any other pharmaceutically acceptable water-soluble or water-swellable material made into beads or pellets. The cores may be spheres of materials such as sucrose/starch (Sugar Spheres NF), sucrose crystals, or extruded and dried spheres typically comprised of excipients such as microcrystalline cellulose and lactose.

The substantially water-insoluble material in the first layer is generally a “GI insoluble” or “GI partially insoluble” film forming polymer (dispersed or dissolved in a solvent). As examples may be mentioned ethyl cellulose, cellulose acetate, cellulose acetate butyrate, polymethacrylates such as ethyl acrylate/methyl methacrylate copolymer (Eudragit NE-30-D) and ammonio methacrylate copolymer types A and B (Eudragit RL30D and RS30D), and silicone elastomers. Usually, a plasticizer is used together with the polymer. Exemplary plasticizers include: dibutylsebacate, propylene glycol, triethylcitrate, tributylcitrate, castor oil, acetylated monoglycerides, acetyl triethylcitrate, acetyl butylcitrate, diethyl phthalate, dibutyl phthalate, triacetin, fractionated coconut oil (medium-chain triglycerides).

The second layer containing the active ingredient may be comprised of the active ingredient (drug) with or without a polymer as a binder. The binder, when used, is usually hydrophilic but may be water-soluble or water-insoluble. Exemplary polymers to be used in the second layer containing the active drug are hydrophilic polymers such as polyvinylpyrrolidone, polyalkylene glycol such as polyethylene glycol, gelatine, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethyl hydroxyethyl cellulose, acrylic acid polymers, polymethacrylates, or any other pharmaceutically acceptable polymer. The ratio of drug to hydrophilic polymer in the second layer is usually in the range of from 1:100 to 100:1 (w/w).

Suitable polymers for use in the third layer, or membrane, for controlling the drug release may be selected from water insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above.

Optionally, the controlled release layer comprises, in addition to the polymers above, another substance(s) with different solubility characteristics, to adjust the permeability, and thereby the release rate, of the controlled release layer. Exemplary polymers that may be used as a modifier together with, for example, ethyl cellulose include: HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility, such as cellulose acetate phthalate or ammonio methacrylate copolymer and methacrylic acid copolymer, or mixtures thereof. Additives such as sucrose, lactose and pharmaceutical grade surfactants may also be included in the controlled release layer, if desired.

Also provided herein are unit dosage forms of the compositions. In such forms, the composition of the present invention is subdivided into unit dosages containing appropriate quantities of the active component (e.g., and without limitation, a compound of Formula (I) or an ester thereof, or a salt of each thereof). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

### Other Actives

A formulation (or kit) in accordance with the invention may contain, in addition to ibudilast and GGA, or pharmaceutically acceptable salts thereof, one or more additional active agents effective in treating progressive neurodegenerative diseases. Preferably, the active agent is one that possesses a mechanism of action different from that of ibudilast and GGA. Such actives include the combinations for pain listed in US Application No. 20060160843, as well as the active ingredients recognized for treatment for the target diseases. Such active ingredients can be found listed in the FDA's Orange Book, Goodman & Gilman The Pharmacological Basis of Therapeutics, J. Griffith Hardman, L. L. Limbird, A. Gilman, 11th Ed., 2005, The Merck Manual, 18th edition, 2007, and The Merck Manual of Medical Information 2003.

### Animal Models

The ability of compositions of the present invention to treat neurodegenerative diseases or disorders can be evaluated by any of the standard progressive neuropathic disease models known in the art. Examples of such models are described in Animal Models of Neurological Disease: Neurodegenerative Diseases (Neuromethods) by Alan A. Boulton, Glen B. Baker, and Roger F. Butterworth (1992); Handbook of Laboratory Animal Science, Second Edition: Volumes I-III (Handbook of Laboratory Animal Science) by Jann Hau (Editor), Jr., Gerald L. Van Hoosier (Editor). (2004); Animal Models of Movement Disorders by Mark LeDoux (Editor), (2005); and Animal Models of Cognitive Impairment (Frontiers in Neuroscience) (2006) by Edward D. Levin (Editor), Jerry J. Buccafusco (Editor).

### Kits

Also provided herein is a kit containing at least one combination composition of the invention, accompanied by instructions for use

For example, in instances in which each of the drugs themselves are administered as individual or separate dosage forms, the kit comprises ibudilast and GGA, or pharmaceutically acceptable salts thereof, along with instructions for use. The ibudilast and GGA, or pharmaceutically acceptable salts thereof, may be packaged in any manner suitable for administration, solong as the packaging, when considered along with the instructions for administration, clearly indicates the manner in which each of the drug components is to be administered.

For example, in an illustrative kit comprising ibudilast and GGA, or pharmaceutically acceptable salts thereof, the kit may be organized by any appropriate time period, such as by day. As an example, for Day 1, a representative kit may comprise unit dosages of each of ibudilast and GGA, or pharmaceutically acceptable salts thereof. If each of the drugs is to be administered twice daily, then the kit may contain, corresponding to Day 1, two rows of unit dosage forms of each of ibudilast and GGA, or pharmaceutically acceptable salts thereof, along with instructions for the timing of administration. Alternatively, if ibudilast and GGA, or pharmaceutically acceptable salts thereof, differ in the timing or quantity of administration, then such would be reflected in the packaging and instructions. Various embodiments according to the above may be readily envisioned, and would of course depend upon the particular combination of drugs, in addition to ibudilast and GGA, or pharmaceutically acceptable salts thereof, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister packs, dessicants, and the like.

It is to be understood that while the invention has been described in conjunction with preferred specific embodiments, the foregoing description as well as the examples that follow are intended to illustrate and not limit the scope of the invention. Other aspects, advantages and modifications within the scope of the invention will be apparent to those skilled in the art to which the invention pertains.

All references mentioned in this application, including any patents, published patent applications, books, handbooks, journal publications, or the FDA Orange Book are hereby incorporated by reference herein, in their entirety.

### EXAMPLES

### Example 1

### Drosophila Life Span Assay as an ALS Treatment Model

Drosophila males will be collected. Flies will be transferred to fresh food (with active compounds) every 2-3 days. Daily, the number of living flies are analyzed. The experiment is performed under temperature controlled conditions (25° C.) and uses negative control (only solvent), and positive controls (wt stock, any antioxidant compound reported as able to increase life span in this fly model). In order to compare the activity of the testing compounds with riluzole (an FDA-approved drug for ALS), this drug will be added to the assay.

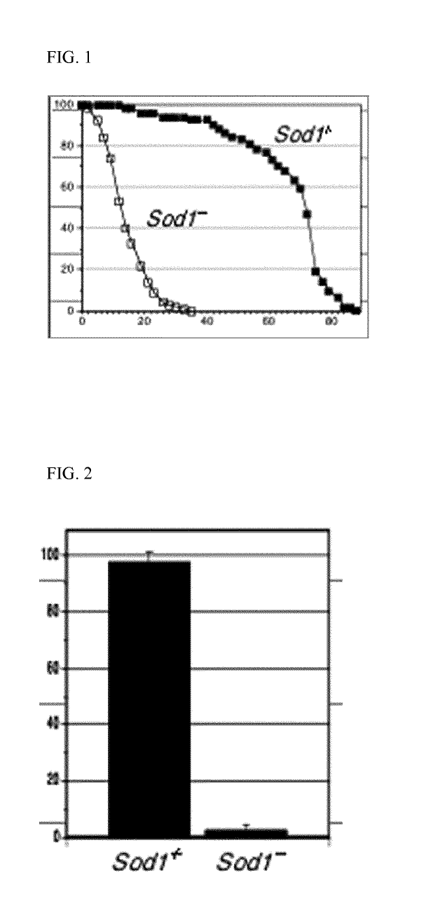

The experiment includes the analysis of different compound concentrations of ibudilast and GGA, each at different concentrations and will evaluate 240 flies for each concentration. Recovering on life span curve can indicate positive compound activity. See, FIG. 1.

Timing: 5 months (1-2 months to expand the fly stock and 3 months for assay execution and results interpretation).

### Example 2

### Drosophila paraquat sensitivity assay as an ALS treatment model

Drosophila males will be collected and keep on fly food for 24 h. Then flies will be transferred to vials containing 3-mm paper filter disks saturated with 250 μl of 1% sucrose containing 2 mM paraquat or 1% sucrose, 2 mM paraquat and the tested compounds. The vials will be stored at 25° C. in the dark, and flies are enumerated after 24 h.

Three replicas for each concentration will be performed in the same day and three replicas of the assay will be performed in different days. A negative control (only solvent), and positive controls (wt stock, any antioxidant compound reported as able to increase life span in this fly model), and riluzole will be added to the assay.

The experiment will test different compound of ibudilast-GGA combinations and will evaluate 360 flies for each concentration. Resistance to paraquat treatment will be indicative of positive activity of the combinations tested. See, FIG. 2.

Timing: 10 weeks (1-2 months to expand the fly stock, two weeks for assay execution and results interpretation)

### Example 3

### Evaluation of Anti-ALS Activity on VAP-33A Drosophila Mutants

From other mutant stocks available and involving other ALS linked genes, loss of function of Vap-33-1 gene (excision of transcribed sequence and loss of protein function) displays valid fly phenotypes for evaluation of compounds activity. Indistinctly, Vap-33A^{Δ448} or Vap-33A^{Δ20} stocks display neurophysiology defects linked to a lethal phenotype during larvae development.

### Viability Assay

Vap-33A^{Δ} mutants are larval lethal with rare adult escapers (˜1%)⁷. Embryos or larvae at stage 1 will be seeded on fly food with different compound concentrations of ibudilast-GGA combinations. Three replicas for each combination will be performed in the same day. Three replicas of the assay will be performed in different days. Number of adult escapers will be quantified after 14 days of compound treatment. A negative control (only solvent), and positive controls (wt stock, any antioxidant compound reported as able to increase life span in this fly model), and riluzole will be added to the assay.

The experiment includes the analysis of four compound combinations and will evaluate 180 flies for each concentration (4 replicates×3 days with 15 flies each one).

Timing: 3 months (2 months to expand the fly stock, 1 month for assay execution and results interpretation

### Example 4

### Evaluation of Ibudilast-GGA Combination in a Rat Model of Alzheimer's Disease

A rat animal model for Alzheimer's disease is administered ibudilast and GGA and an increase brain mass is achieved for the group of animals being administered ibudilast, thereby indicating that this model can be effective for the treatment of Alzheimer′ disease in humans.

### Example 5

### Evaluation of Ibudilast-GGA Combination in a Rat Model of ALS

An animal model for ALS is administered ibudilast and an increase brain mass is achieved for the group of animals being administered ibudilast, thereby indicating that this model can be effective for the treatment ALS.

### Example 6

### Evaluation of Ibudilast-GGA Combination in a Rat Model of Parkinson's Disease

An animal model for Parkinson's disease is administered ibudilast and an increase brain mass is achieved for the group of animals being administered ibudilast, thereby indicating that this model can be effective for the treatment Parkinson's disease.

### Example 7

### Clinical Trial of the Ibudilast-GGA Combination to Evaluate its Effectiveness in Alzheimer's Disease

A combination of ibudilast and GGA is administered to patients exhibiting the symptoms of Alzheimer's disease, as diagnosed by their physician and confirmed by an independent board-certified neurologist. Prior to the clinical trial, the patients undergo appropriate psychoneurological tests such as the Mini Mental Status Exam (MMSE), the Alzheimer Disease Assessment Scale (ADAS), the Boston Naming Test (BNT), and the Token Test (TT). Neuropsychological tests are repeated at appropriate points during the clinical trial. The tests are performed by neuropsychologists who are not aware of the patients' treatment regimen.

In this double blind study, patients are randomly assigned to the test group or placebo at the beginning of the study. The ibudilast and GGA, or pharmaceutically acceptable salts thereof, and placebo are administered orally at pre-designated intervals. The test patients are evaluated for a specified period of time to determine the effectiveness of treatment using the composition as compared to the control group individuals given a placebo. Scores are statistically compared between the test composition and the placebo for each of the three observational periods. Without treatment, the natural course of Alzheimer's disease results in significant deterioration of a patient's test scores during the course of the clinical trial. A patient treated with the combination of the invention is considered improved if the patient's scores remain the same or improve compared to placebo during the course of the clinical trial.

### Example 8

### Clinical Trial of the Ibudilast-GGA Combination to Evaluate its Effectiveness in ALS

A combination of ibudilast and GGA, or pharmaceutically acceptable salts thereof, are administered to patients exhibiting the symptoms of ALS. In a double blind study, ibudilast and GGA, or pharmaceutically acceptable salts thereof, and placebo are administered orally at pre-designated intervals to a patient group and a placebo group. The test patients are evaluated for a specified period of time to determine the effectiveness of treatment using the composition as compared to the control group individuals given a placebo. The TUFTS Quantitative Neuromuscular Examination (TQNE) is a well standardized, reliable, validated test to measure strength and function in ALS. The test involves measurement of maximum voluntary isometric contraction (MVIC) of 8 muscle groups in the arms using a strain gauge tensiometer. This measurement is a standard for clinical trials in ALS. The ALS Functional Rating Scale (ALSFRS) is an rating scale used to determine patients' assessment of their ability and independence in 10 functional activities. Validity has been established by correlating ALSFRS scores with change in strength over time. The ALSFRS is generally a secondary outcome measure in clinical trials. A patient treated with the combination of the invention is considered improved if the patient's scores remain the same or improve compared to placebo during the course of the clinical trial.

### Example 9

### Clinical Trial of the Ibudilast-GGA Combination to Evaluate its Effectiveness in Parkinson's Disease

A combination of ibudilast and GGA, or pharmaceutically acceptable salts thereof, are administered to patients exhibiting the symptoms of Parkinson's disease. In a double blind study, the test combination and placebo are administered orally at pre-designated intervals to a patient group and a placebo group. The test patients are evaluated for a specified period of time to determine the effectiveness of treatment using the composition as compared to the control group individuals given a placebo. The prespecified primary efficacy outcome for Parkinson's disease is a change in the Activities of Daily Living and Motor components of the generally accepted Unified Parkinson's Disease Rating Scale (UPDRS II/III) between baseline and the last evaluation on treatment. Other assessment scales such as the UPDRS component scores (mental, motor, ADL), the modified Hoehn and Yahr Stage, Modified Schwab and England ADL score may be used to evaluate the efficacy of the present invention.

### Example 10

### Clinical Trial of the Ibudilast-GGA Combination to Evaluate its Effectiveness in Multiple Sclerosis

A combination of ibudilast and GGA, or pharmaceutically acceptable salts thereof, are administered to patients exhibiting the symptoms of multiple sclerosis. In a double blind study, the test composition and placebo are administered orally at pre-designated intervals to a patient group and a placebo group. A clinical trial will include multiple sclerosis patients diagnosed on McDonald criteria, with a baseline Expanded Disability Status Scale (EDDS) between 0 and 5 and either at least one relapse within the last 12 months of randomization and a previous MRI scanning showing lesions consistent with multiple sclerosis or Gd E lesions on MRI scan done within 6 months of randomization.

The primary endpoint for the clinical trial is time-to-confirmed disease progression or treatment failure as measured by EDSS or Multiple Sclerosis Functional Composite Score. Secondary endpoints include relapse rate-related endpoints and MRI measurement-related endpoints. Other tertiary endpoints may be measured, including cognitive function-related endpoints and quality of life-related endpoints.

What is claimed is:

1. A method of alleviating negative effects of a neurodegenerative disease or disorder in a human patient suffering therefrom, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

2. The method of claim 1, wherein the ibudilast and GGA, or pharmaceutically acceptable salts thereof, are administered in separate dosage forms.

3. The method of claim 1, wherein ibudilast and GGA, or pharmaceutically acceptable salts thereof, are administered in the same dosage form.

4. The method of claim 1, in which the ibudilast and GGA, or pharmaceutically acceptable salts thereof, are administered orally.

5. The method of claim 1, in which the ibudilast and GGA, or pharmaceutically acceptable salts thereof, are administered in a tablet or a capsule dosage form.

6. The method of claim 1, in which the ibudilast and GGA, or pharmaceutically acceptable salts thereof, are administered in a liquid dosage form.

7. The method of claim 1, wherein the ibudilast or a pharmaceutically acceptable salt thereof is administered in an amount from about 100 mg/day to about 4,000 mg/day, divided into one, two, or three portions.

8. The method of claim 1, in which the GGA or a pharmaceutically acceptable salt thereof is administered in an amount from about 1 mg/kg/day to about 1000 mg/kg/day of the patient, divided into one, two, or three portions.

9. The method of claim 1, wherein the neurodegenerative disease or disorder compromises the nervous system.

10. The method of claim 1, wherein the neurodegenerative disease or disorder is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, primary lateral sclerosis, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, but not relapsing, remitting multiple sclerosis, frontotemporal dementia, Wilson's disease, progressive neuropathic pain, ischemia caused by stroke, traumatic brain injury, or spinal cord injury.

11. The method of claim 1, wherein the neurodegenerative disease or disorder is Alzheimer's disease, Parkinson's disease, amyotrophic lateral sclerosis (ALS), or multiple sclerosis.

12. The method of claim 1, wherein the ibudilast and the GGA, or pharmaceutically acceptable salts thereof, are administered simultaneously.

13. The method of claim 1, wherein the ibudilast and the GGA, or pharmaceutically acceptable salts thereof, are administered consecutively.

14. A method of slowing progression of disease in a patient diagnosed with a chronic neurodegenerative disease, comprising administering to the patient:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

15. The method of claim 14, in which the ibudilast and GGA, or pharmaceutically acceptable salts thereof, are administered orally.

16. The method of claim 14, in which the ibudilast and GGA, or pharmaceutically acceptable salts thereof, are administered in a tablet or a capsule dosage form.

17. The method of claim 14, in which the ibudilast and GGA, or pharmaceutically acceptable salts thereof, are administered in a liquid dosage form.

18. The method of claim 14, wherein the ibudilast or a pharmaceutically acceptable salt thereof is administered in an amount from about 100 mg to about 4,000 mg/day, divided into one, two, or three portions.

19. The method of claim 14, in which the GGA or pharmaceutically acceptable salt thereof is administered in an amount from about 1 mg/kg/day to about 1000 mg/kg/day of the patient.

20. The method of claim 14, wherein the neurodegenerative disease compromises the nervous system.

21. The method of claim 14, wherein the neurodegenerative disease is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, primary lateral sclerosis, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, but not relapsing, remitting multiple sclerosis, frontotemporal dementia, Wilson's disease, progressive neuropathic pain, ischemia caused by stroke, traumatic brain injury, or spinal cord injury.

22. The method of claim 14, wherein the neurodegenerative disease or disorder is Alzheimer's disease, Parkinson's disease, amyotrophic lateral sclerosis (ALS), or multiple sclerosis.

23. The method of claim 14, wherein the ibudilast and the GGA are administered simultaneously.

24. The method of claim 14, wherein the ibudilast and the GGA are administered consecutively.

25. A composition for oral administration, comprising:
(a) ibudilast or a pharmaceutically acceptable salt thereof,
(b) GGA or a pharmaceutically acceptable salts thereof, and
(c) optionally, a pharmaceutically acceptable excipient or carrier.

26. The composition of claim 25, comprising ibudilast or a pharmaceutically acceptable salt thereof in an amount from about 100 mg to about 4,000 mg, divided into one, two, or three portions.

27. The composition of claim 25, comprising from about 10 to 4,000 mg of GGA or a pharmaceutically acceptable salt thereof, divided into one, two, or three portions.

28. The composition of claim 25, comprising from about 100 mg to about 4,000 mg of ibudilast or a pharmaceutically acceptable salt thereof, and from about 10 to 4,000 mg of GGA or a pharmaceutically acceptable salt thereof.

29. The composition of claim 25, wherein the ibudilast and the GGA, or
pharmaceutically acceptable salts thereof, are in a single tablet or a single capsule dosage form.

30. The composition of claim 25, wherein the ibudilast and the GGA, or
pharmaceutically acceptable salts thereof, are in a liquid dosage form.

31. A method of treating a patient diagnosed with a neurodegenerative disease or disorder, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof.

32. The method of claim 31, in which the ibudilast and GGA, or pharmaceutically acceptable salts thereof, are administered orally.

33. The method of claim 31, in which the ibudilast and GGA, or pharmaceutically acceptable salts thereof, are administered in a tablet or a capsule dosage form.

34. The method of claim 31, in which the ibudilast and GGA, or pharmaceutically acceptable salts thereof, are administered in a liquid dosage form.

35. The method of claim 31, wherein the ibudilast or a pharmaceutically acceptable salt thereof is administered in an amount from about 100 mg to about 4,000 mg/day, divided into one, two, or three portions.

36. The method of claim 31, in which the GGA or pharmaceutically acceptable salt thereof is administered in an amount from about 1 mg/kg/day to about 1000 mg/kg/day of the patient, divided into one, two, or three portions.

37. The method of claim 31, wherein the neurodegenerative disease or disorder compromises the nervous system.

38. The method of claim 31, wherein the neurodegenerative disease or disorder is Alzheimer's disease, Senile dementia of the Alzheimer type, Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, corticodentatinigral degeneration, Hallervorden-Spatz disease, progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, Gilles de la Tourette syndrome, progressive ataxia, cerebellar degenerations, spinocerebellar degenerations, cerebellar cortical degeneration, olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), other forms of familial spinal muscular atrophy, primary lateral sclerosis, hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, motor neuron disease, progressive ataxias, primary lateral sclerosis, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis muliplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, Alper's disease, primary progressive or secondary progressive multiple sclerosis, but not relapsing, remitting multiple sclerosis, frontotemporal dementia, Wilson's disease, progressive neuropathic pain, ischemia caused by stroke, traumatic brain injury, or spinal cord injury.

39. The method of claim 31, wherein the neurodegenerative disease or disorder is Alzheimer's disease, Parkinson's disease, amyotrophic lateral sclerosis (ALS), or multiple sclerosis.

40. The method of claim 31, wherein the ibudilast and the GGA, or pharmaceutically acceptable salts thereof, are administered simultaneously.

41. The method of claim 31, wherein the ibudilast and the GGA, or pharmaceutically acceptable salts thereof, are administered consecutively.

42. A method of reducing a volume of an infarct (an area of necrosis in a tissue or organ resulting from obstruction of the local circulation by a thrombus or embolus) in a patient suffering from an ischemia, comprising administering to a patient in need thereof:
(a) a therapeutically effective amount of ibudilast or a pharmaceutically acceptable salt thereof, and
(b) a therapeutically effective amount of geranylgeranylacetone (GGA) or a pharmaceutically acceptable salt thereof, in which a volume of an infarct in the treated patient is reduced compared to a volume of an infarct in a control patient.

43. The method of claim 42, in which the ibudilast and GGA, or pharmaceutically acceptable salts thereof, are administered orally.

44. The method of claim 42, in which the ibudilast and GGA, or pharmaceutically acceptable salts thereof, are administered in a tablet or a capsule dosage form.

45. The method of claim 42, in which the ibudilast and GGA, or pharmaceutically acceptable salts thereof, are administered in a liquid dosage form.

46. The composition of claim 42, wherein the ibudilast or a pharmaceutically acceptable salt thereof is administered in an amount from about 100 mg to about 4,000 mg/day, divided into one, two, or three portions.

47. The method of claim 42, in which the GGA or a pharmaceutically acceptable salt thereof is administered in an amount from about 1 mg/kg/day to about 1000 mg/kg/day of the patient, divided into one, two, or three portions.

48. The method of claim 42, wherein the ibudilast and the GGA, or pharmaceutically acceptable salts thereof, are administered simultaneously.

49. The method of claim 42 or any preceding claim, wherein the ibudilast and the GGA, or pharmaceutically acceptable salts thereof, are administered consecutively in any order.
